(19) United States
(12) Patent Application Publication
(10) Pub. No.: US 2011/0082326 A1
(43) Pub. Date: Apr. 7, 2011

(54) TREATMENT OF CLINICAL APPLICATIONS WITH NEUROMODULATION

(76) Inventors: David J. Mishelevich, Playa del Rey, CA (US); M. Bret Schneider, Portola Valley, CA (US)

(21) Appl. No.: 12/917,236

(22) Filed: Nov. 1, 2010

Related U.S. Application Data
(63) Continuation-in-part of application No. 12/402,404, filed on Mar. 11, 2009.
Application No. 12/402,404 is a division of application No. 10/821,807, filed on Apr. 9, 2004, now Pat. No. 7,520,848.
Application No. 10/821,807 is a continuation-in-part of application No. 11/429,504, filed on May 5, 2006.
(60) Provisional application No. 61/256,480, filed on Oct. 30, 2009.

(51) Int. Cl. A61N 2/02

(52) U.S. Cl. 600/13; 600/15

(57) ABSTRACT

Described herein are systems and methods for Transcranial Magnetic Stimulation (TMS) including one or more TMS electromagnets for stimulation of target deep brain regions to stimulate, enhance and/or inhibit neural activity.

### CROSS REFERENCE TO RELATED APPLICATIONS

This patent application claims priority as a continuation-in-part of U.S. patent application Ser. No. 12/402,404, filed Mar. 11, 2009 and titled “ROBOTIC APPARATUS FOR TARGETING AND PRODUCING DEEP, FOCUSED TRANSCRANIAL MAGNETIC STIMULATION,” which is a divisional of U.S. patent application Ser. No. 10/821,807, filed Apr. 9, 2004, now U.S. Pat. No. 7,520,848 and titled “ROBOTIC APPARATUS FOR TARGETING AND PRODUCING DEEP, FOCUSED TRANSCRANIAL MAGNETIC STIMULATION.”

This patent application also claims priority as a continuation-in-part to U.S. patent application Ser. No. 11/429,504, filed on May 5, 2006 and titled “TRAJECTORY-BASED DEEP-BRAIN STEREOTACTIC TRANSCRANIAL MAGNETIC STIMULATION”.

This application also claim priority to provisional patent application Ser. No. 61/256,480, filed on Oct. 30, 2009 and titled “TREATMENT OF CLINICAL APPLICATIONS WITH NEUROMODULATION.” The disclosures of each of these patent applications are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are systems and methods for Transcranial Magnetic Stimulation (TMS) including one or more TMS electromagnets for stimulation of target deep brain regions to stimulate, enhance and/or inhibit neural activity.

### BACKGROUND OF THE INVENTION

A variety of techniques have been developed for neuromodulation of neural structures, including Transcranial Magnetic Stimulation (TMS), Deep Brain Stimulation (DBS), open surgery, Stereotactic Radiosurgery, transcranial Direct Current Simulation (tDCS), and ultra sound. There are functional as well as technical differences between these different modalities. For example, stereotactic Transcranial Magnetic Stimulation (sTMS) allows direct neuromodulation of deep targets while traditional repetitive Transcranial Magnetic Stimulation (rTMS) does not. Some modalities are non-invasive, including TMS, ultrasound, tDCS, and ionizing radiation. Other modalities are at least somewhat invasive, including DBS, open surgery, surface electrodes, optogenetic, and Stereotactic Radiosurgery. Examples of other modalities are described in US 2009/0112133 and US 2009/0114849.

Further, some targets may be impractical for modulation with a particular modality. For example, the Insula is believed to be too vascular to allow treatment using traditional DBS. The identification of suitable targets, both individually and as elements of neural circuits, for a particular modality is of fundamental importance. The identification of configurations allowing treatment is of particular importance.

In particular, the stimulation of deep brain targets, of combinations of neuronal targets including deep brain targets, of multiple superficial targets, and/or of combinations of superficial and deep brain targets has traditionally been difficult or impossible to achieve by Transcranial Magnetic Stimulation (TMS). The methods, systems and devices for deep-brain stimulation using TMS that we have previously described (see, e.g., the references incorporated in their entirety above), may allow for specific and meaningful targeting of such targets. Described below are systems, devices and methods for modulating these targets in a manner that was previously not possible.

### SUMMARY OF THE INVENTION

Neuromodulation of target neural structures by up-regulating or down-regulating their activity results in treatment of clinical conditions/applications. Described herein are methods of non-invasively modulating specific identified target structures believed to be involved in neural circuits that may be therapeutically important. In particular, described herein are Transcranial Magnetic Stimulation (TMS) devices, systems and methods for modulating these targets. In general, the TMS devices, methods and systems described herein are configured to stimulate deep brain targets (such as those referred to above) or combinations of targets including deep brain targets.

These brain targets may comprise neural circuits, as described in greater detail below. The targets (e.g., portions of the circuit) may be discrete neuroanatomical regions (e.g., target regions of the brain) that are groups of neurons that are organized into anatomical bodies. Thus, the targets may be neuroanatomical structures within the brain. Neuroanatomical structures may have discrete functions and may be organized by morphology. Exemplary neuroanatomic structures may include NeoCortex, Medial PFC, LDLPFC, RDLPFC, Dorsomedial PFC, Ventral PFC, VMPFC, Orbitofrontal Cortex (OFC), Tracts between OFC and Insula, Cingulate Genu, DACG, Pre-Gen. Anterior Cingulate, Subgenual Cingulate, Posterior Cingulate, Striatum-DACG Connections, Tracts between Pre-Gen. Anterior Cingulate and Insula, Insula, Amygdala, Anterior Limb of Internal Capsule, Ventral Internal Capsule, Target, Nucleus Accumbens, Tract between Nucleus Acumbens and Ventral Teg., Hippocampus, Temporal Lobes, Septum, Caudate (Nucleus), Globus Pallidus, Anterior Nucleus of the Thalamus, Lateral Thalamus, Centromedian Thalamus, Thalamic Subregions, Subthalamic Nucleus, Lateral Hypothalamic Area Nuclei, Ventromedial Nuclei of Hypothal., Cerebellum, Brainstem, and Pons.

The TMS systems described herein (which may also be referred to as “deep-brain TMS systems”) typically included electromagnetic coils and/or coil arrays configured to treat clinical applications.

As referred to herein, “up-regulation” (or “up”) refers to neuromodulation to increase the level of neural firing and/or metabolic activity in the targeted brain region. Up-regulation is usually accompanied by (or correlated with) an increase in blood flow to the up-regulated region of the brain. In general, up-regulation may mean an increase in action potentials fired in the region up-regulated, as well as an increase in glucose consumption. Down-regulation typically means the opposite of up-regulation, and may mean suppression of metabolic activity in the region. Thus, in a down-regulated region there may be fewer spontaneous action potential firings (i.e., a reduction in action potential firings).

The tables shown in FIGS. 8 and 9 illustrate exemplary application of up-regulation and down-regulation.

### BRIEF DESCRIPTION OF THE DRAWINGS

FIG. 1A is a diagram of a neural circuit for addiction.

FIG. 1B is a diagram of a neural circuit for pain.

FIG. 2 is a diagram of a neural circuit for depression.

FIG. 3 is a diagram of a neural circuit for obesity.

FIG. 4 is a diagram of a neural circuit for Obsessive Compulsive Disorder (OCD).

FIGS. 5, 6, and 7 illustrate a selected set of electromagnetic coil configurations.

FIGS. 8 and 9 illustrate selected applications mapped onto targets and associated electromagnetic coil configurations which may be used in their treatment as described herein.

FIG. 10 is a table of three alternative coil configurations for the treatment of Addiction.

FIG. 11 is a table of four alternative coil configurations for the treatment of depression.

FIG. 12 is a table of three alternative coil configurations for the treatment of pain.

FIG. 13 is a CAD illustration of one variation of TMS electromagnets configured to treat Addiction.

FIG. 14 is a CAD illustration of another variation of TMS electromagnets configured to treat Addiction.

FIG. 15 is a CAD illustration of another variation of TMS electromagnets configured to treat Addiction.

FIG. 16 is a CAD illustration of one variation of TMS electromagnets configured to treat depression.

FIG. 17 is a CAD illustration of another variation of TMS electromagnets configured to treat depression.

FIG. 18 is a CAD illustration of another variation of TMS electromagnets configured to treat depression.

FIG. 19 is a CAD illustration of another variation of TMS electromagnets configured to treat depression.

FIG. 20 is a CAD illustration of one variation of TMS electromagnets configured to treat pain.

FIG. 21 is a CAD illustration of another variation of TMS electromagnets configured to treat pain.

FIG. 22 is a CAD illustration of another variation of TMS electromagnets configured to treat pain.

FIG. 23 shows interdigitated figure-8 coils.

### DETAILED DESCRIPTION OF THE INVENTION

In general, the systems, devices and methods described herein and incorporated by reference may be used to treat one or more clinical conditions. Although the methods, devices and systems described herein are directed primarily to Transcranial Magnetic Stimulation (TMS), some of the principles described herein may be applied or adapted for use with other modalities, or in combination with other modalities. For example, a particular clinical application or condition may be impacted by a therapeutic modality based on the impact of that modality on one or more neural targets. This is true whether the modality is TMS, DBS, ultrasound, stereotactic radiosurgery or other treatment.

Physiological functions and, if there are problems, clinical conditions are usually not controlled by a single neural structure, but a neural circuit. The circuit is typically made up of neural structures (e.g., nuclei and tracts, gray matter, white matter, etc.) each of which is a potential target for therapeutic intervention. However, not all potential targets are practical targets. A given potential target may be in accessible (e.g., too deep to be reached by a given therapeutic modality), may have critical structures that might be negatively impacted by a therapeutic modality in close proximity and thus be too dangerous to attempt, or may be impractical because targeting that structure may physically prevent targeting a higher-priority structure.

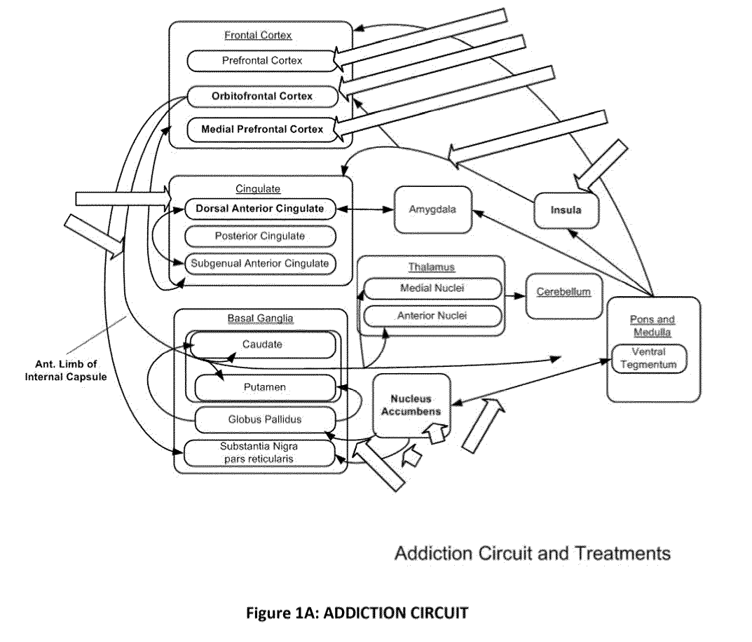

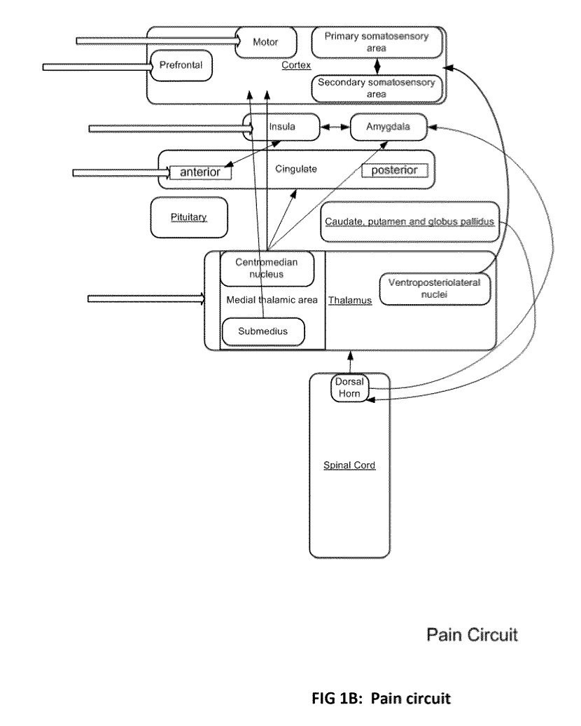

For example, FIG. 1A shows a schematic diagram a neural circuit for addiction. From this circuit, potential targets include the Prefrontal Cortex, Orbitofrontal Cortex, Medial Prefrontal Cortex, DACG, Subgenual Anterior Cingulate, Nucleus Accumbens, Tract between the Nucleus Accumbens and Ventral Tegmentum, Insula, and the Tract between the Insula, and the Orbitofrontal Cortex. A subset of these targets may be chosen for therapy. Which subset is practical will depend on the therapeutic modality.

As described in greater detail herein, stimulation of one or more targets (including combinations of these targets) may be made possible using the TMS devices and systems for deep brain stimulation described herein in a way that would not otherwise be possible by traditional TMS. FIG. 1B shows a similar neural circuit for pain. In any of the neural circuit diagrams provided (e.g., FIGS. 1A-4), the two-dimensional arrows point toward targets that may be modulated to treat a disorder affiliated with the circuit.

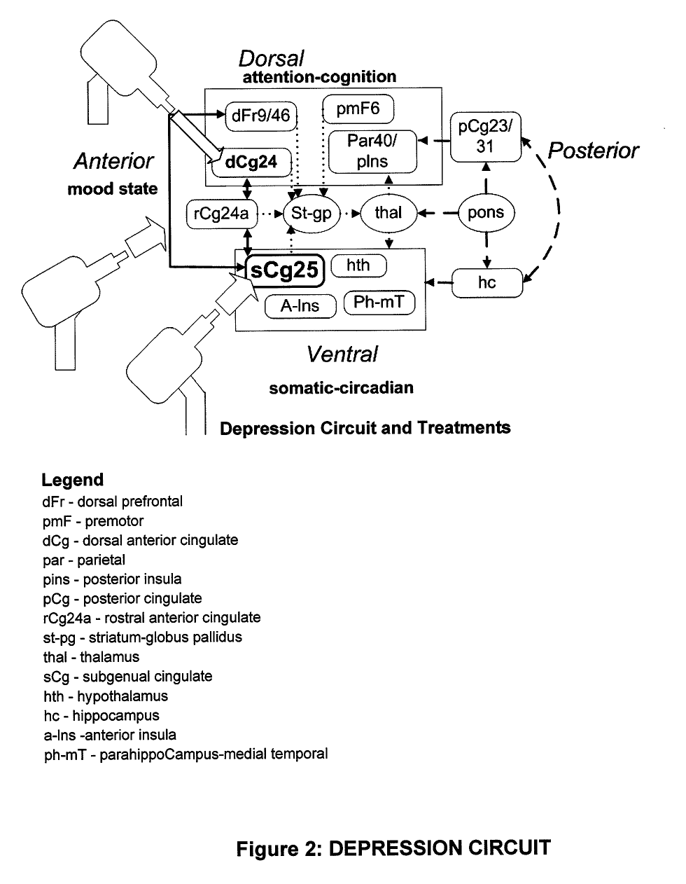

FIG. 2 shows a neural circuit for depression. This neural circuit suggests that potential targets include the Doral Anterior Cingulate (Brodmann's Area 24), the Rostral Anterior Cingulate (Brodmann's Area 24a), and the Subgenual Cingulate (Brodmann's Area 25).

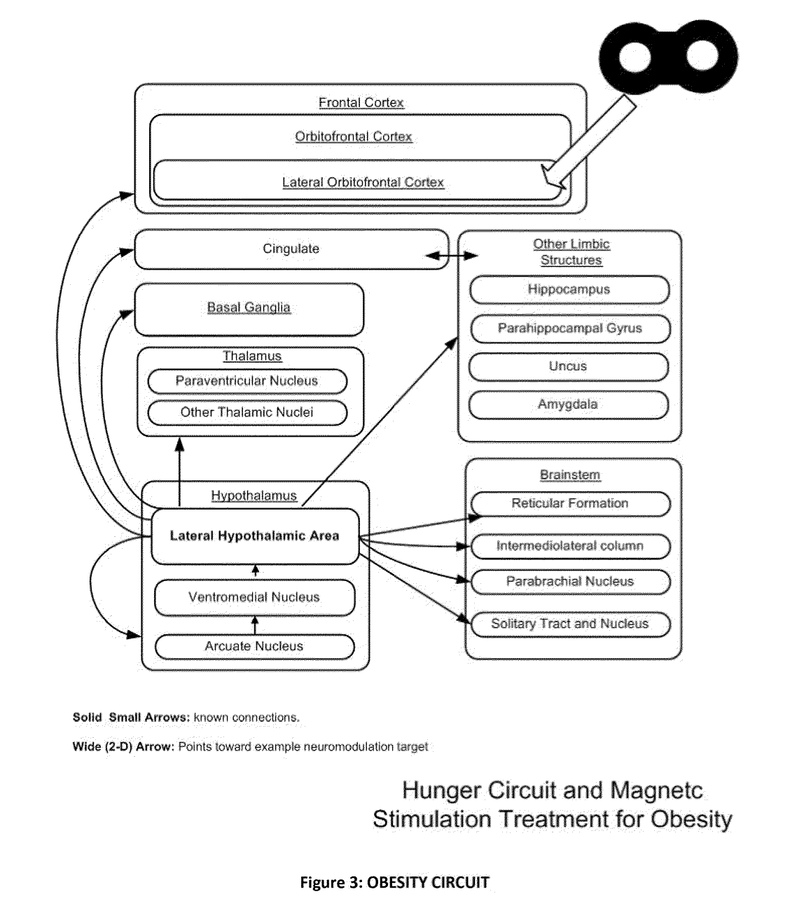

Similarly, FIG. 3 shows a neural circuit for obesity. This circuit shows a particular single potential target, the Lateral Orbitofrontal Cortex. This target is of particular interest because the Oribitofrontal Cortex is the final pathway for output of the Lateral Hypothalamic Area where hunger sensation is transmitted. By down-regulation of the Lateral Hypothalamic Area; perceived hunger may be decreased. The same effect may be achieved by down-modulation of the Oribito-Frontal Cortex target instead, a target that is much more accessible and thus much easier to stimulate.

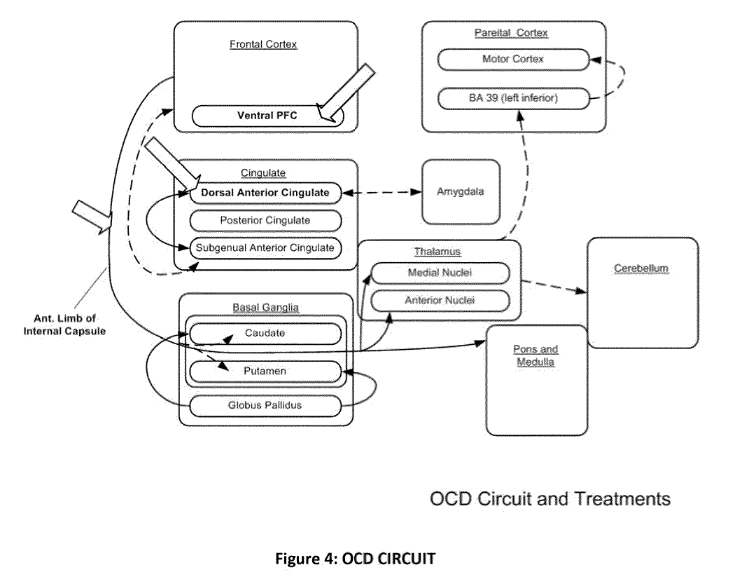

FIG. 4 shows a neural circuit for OCD. Potential targets in this circuit include the Ventral Prefrontal Cortex, the Anterior of the Internal Capsule, and the Dorsal Anterior Cingulate.

The methods described herein include modulating multiple targets in a particular neural circuit in order to affect a clinical result. For example, hitting (modulating) multiple targets in a neural circuit may facilitate Long-Term Potentiation (LTP) or Long-Term Depression (LTD) and thus result in a more durable treatment. Modulating a target typically means having energy reach the target so that it is either up-regulated or down-regulated. Modulation a target may also mean that the target structure is modulated without substantially modulating non-target region. The TMS systems described herein may be configured to hit (modulate) multiple targets. Hitting multiple targets (particularly in a coordinated manner) may improve therapeutic effectiveness by modulating multiple points in the neural circuit, potentially allowing the therapeutic effect to be achieved in a shorter period of therapy time. While alternative therapeutic modalities may used to hit multiple targets, some modalities may be less suitable to hitting multiple targets. For example, using DBS to hit Orbitofrontal Cortex, Dorsal Anterior Cingulate, and the Insula simultaneously would be both very risky and very expensive. The Insula, because of its vascularity, is typically not considered a target for DBS.

As described herein, the methods of TMS described may be used to stimulate multiple sites within a circuit (including deep-brain sites) in a coordinated fashion. In particular the method of TMS stimulation described herein may be used to stimulate one or more targets within a neural circuit to modulate the circuit and thereby effect a change in the particular indication controlled by the circuit. For example, in one exemplary neural circuit having three target sites, LTP or LTD may be achieved. Typically, LTP will involve higher stimulation powers, synchronous stimulation (hit all the three targets at the same time), and faster stimulation rates at all three target sites. LTD, on the other hand, typically might involve lower stimulation powers, asynchronous stimulation (hit the three targets at different times), and lower stimulation rates at the different target sites. The TMS systems and devices described herein may be used to achieve LTP or LTD in such circuits.

### Stereotactic Transcranial Magnetic Stimulation

Stereotactic Transcranial Magnetic Stimulation (sTMS) may be used to hit multiple targets using multiple arrays. These arrays can positively interact in that a coil with one array may substitute for a coil that would normally occur in another array. This can be helpful because there physically may not be enough room for both the single coil serving the two arrays and the coil that was replaced by that single coil. The multiple targets may be located at a mixture of deep and superficial locations or may be all one or the other. The multiple targets may be pulsed simultaneously or the pulses may be interleaved.

Techniques that are applicable to multiple-coil arrays used for Stereotactic Transcranial Magnetic Stimulation (sTMS) include:
The application of pulse patterns where coils are not necessarily stimulated simultaneously.
The use of a single stimulator to energize multiple TMS electromagnets. While typically each coil is pulsed by a single stimulator, in some cases a single stimulator may pulse two or more magnets in the same array or in different arrays. Thus, the number of required stimulators can be reduced. A different array may be defined as one that is aimed toward a different target.
sTMS can be accomplished with sub-Motor-Threshold stimulation at target or higher-powered stimulation.
Power levels may be adjusted related to depth of target and patient-specific factors.
Real-time feedback from patient may be used to modify stimulation parameters including coil position.
Enhanced perturbations.
   TMS (sTMS) Configurations for Clinical Applications

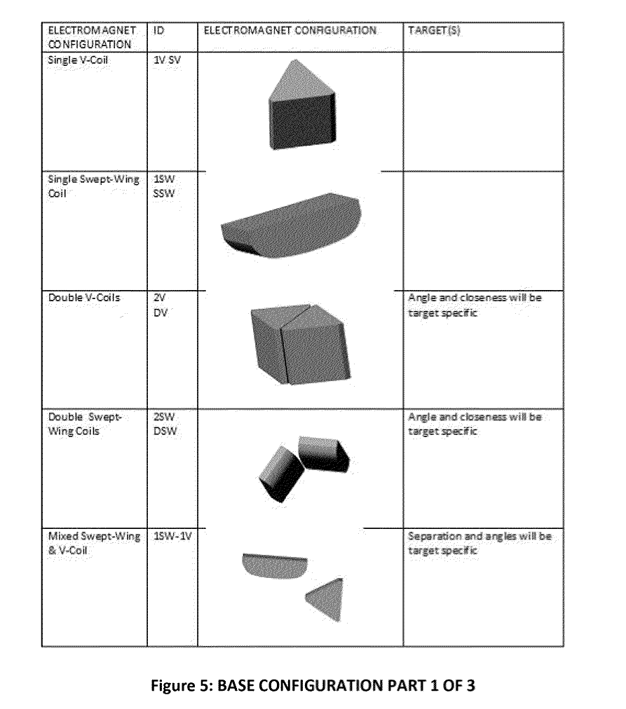

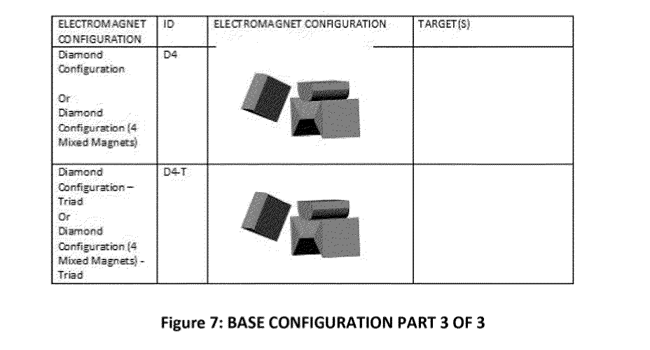

The treatment of various medical conditions using Transcranial Magnetic Stimulation as described herein may depend on the configurations of the electromagnetic coils and how they are positioned. Exemplary electromagnetic coil configurations (including arrays of TMS electromagnets) are shown in FIGS. 5-7. These are not meant to be exclusive. In these figures, the V-coil and Swept-Wing coils shown as examples were custom developed to specifications. Such coils may be powered by a stimulator such as the Magstim Rapid™ stimulator, or any other appropriate stimulator.

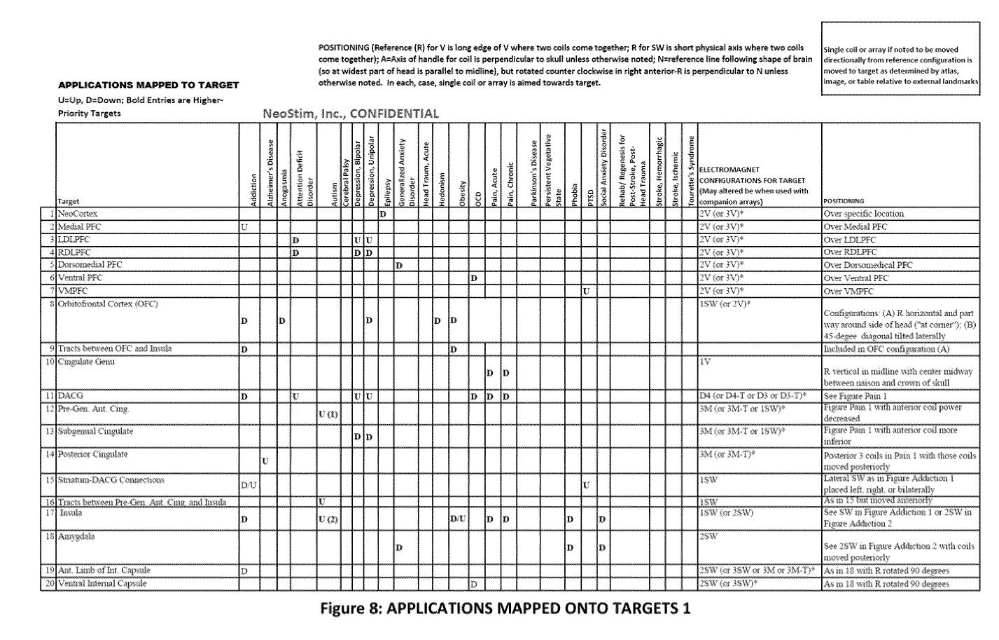

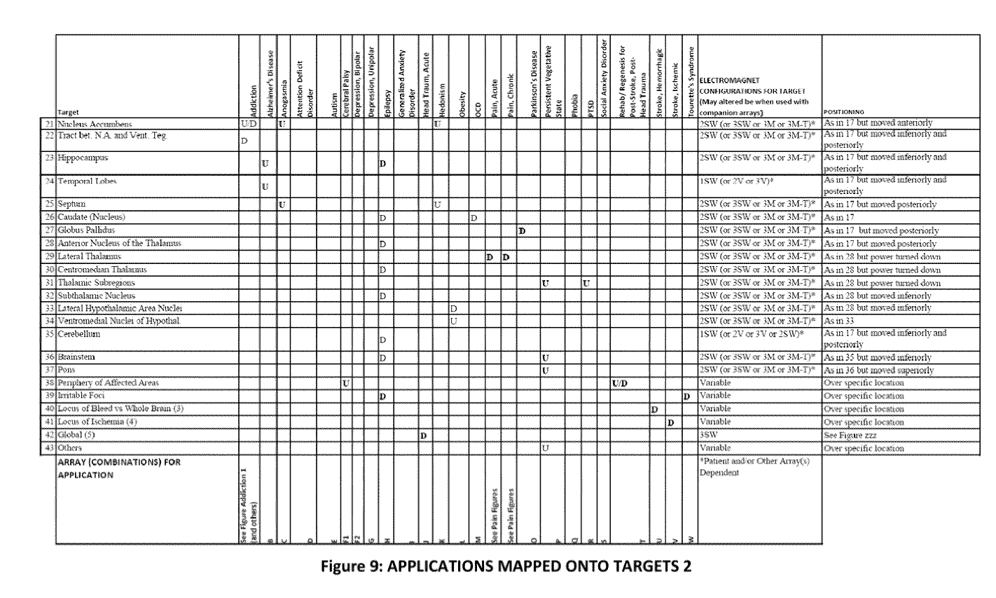

The tables show in FIGS. 8 and 9 illustrate various exemplary configurations for a selection of clinical applications. Electromagnetic coil configurations shown represent a selection and the invention is not limited to these particular configurations. Other configurations of arrays of TMS electromagnets may be used, including configurations not described in the examples shown in FIGS. 5-7.

Global stimulation, say for Acute Head Trauma, may be achieved by using a three swept-wing-coil configuration over the top of the head combined with two swept-wing-coil configurations placed parallel to it, one located anteriorly over the forehead aimed inward and the other located posteriorly over the posterior of the head aimed inward. While generally the targets may be nuclei or cortical regions, tracts (especially long ones), or a combination of a non-tract plus a tract (e.g., Orbitofrontal Cortex combined with the tract between the OFC and the Insula) are important targets as well. The effectiveness of sTMS on a target may be increased proportional to the size of the target (e.g., a bigger target may function as a larger “antenna”). The size of a target can be effectively increased by including tracts that are associated with a target, particularly with those providing connections with associated target structures. Thus non-tracts plus tracts may offer a larger antenna. A consequence is that if one has, for example, three interconnected non-tracts, one can have three non-tract-tract-non-tract target pairs.

Alternative configurations may be applicable for a given target, and the selection of which one is used may be dependent on what other single coils or arrays may be used in the case of multiple targets being hit for a given clinical application. Both physical constraints and functional interactions among coils are applicable and drive the choice, in some cases resulting in a logical choice among alternatives. Aiming of a given configuration may also be patient dependent. In some cases, the configuration may need to be reversed. Targeting a given target, if large, may need to be more specific for a given application, like superior anterior Insula for addiction. While a given application might have multiple targets associated with it, it will not always be desirable to hit the maximum number of targets possible. Selection of a subset may reduce undesirable side effects and may be, like drugs, patient specific. Another consideration is how many TMS stimulators would be required.

The triad configurations (in which lateral electromagnets have their drive currents in opposite polarity to the central coil) can be used on a patient-specific basis instead of the same physical configuration where all of the coils have their drive currents flowing in the same direction.

Positions of the coils may be determined by using one of more of atlas (e.g., the Tailarach Atlas used in neurosurgery), imaging (e.g., PET or fMRI), or tables containing positions of targets with respect to external landmarks on head.

The coils may be held in place relative to each other and positioned relative to the patient's head by coil-fixation devices. These positions could be positioned manually or robotically. The manual position could be a cradle with pockets to place the coils at the correct locations and orientations. In some variations, one or more coils may be moved to achieve stimulation.

### Coil Placements for Addiction, Depression, and Pain

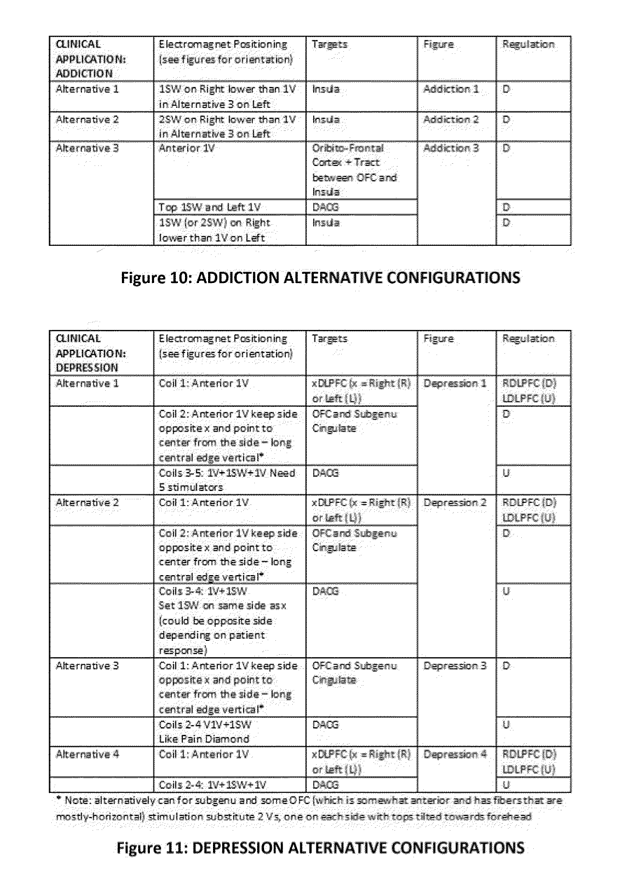

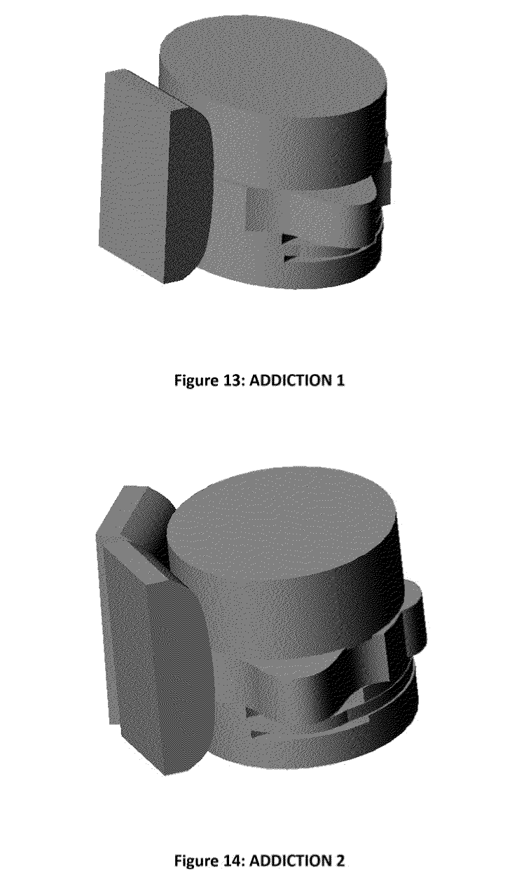

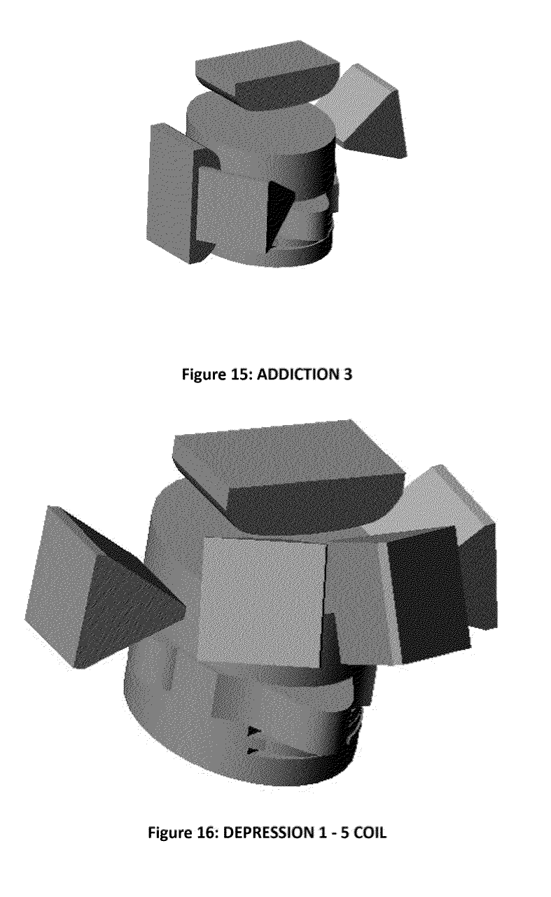

Three alternatives for coil configurations for Addiction are included in the table that appears in FIG. 10. As noted, drawings of these alternatives appear in FIGS. 13, 14, and 15. In all the references, “right” and “left” refer to the patient's right and left. In FIG. 13. FIGS. 13-22 show different arrangements and configurations of TMS electromagnets around a crude simulation of a patient's head. Alternative 1 employs a single Swept Wing Coil with its long axis vertical to down-regulate the Insula. In FIG. 14, Alternative 2, a two Swept-Wing Coil configuration is substituted supporting deeper penetration of the magnetic field. In FIG. 15, Alternative 3, four targets are addressed: the Orbito-Frontal Cortex (OFC), the tract between the OFC and the Insula, the Dorsal Anterior Cingulate Gyrus (DACG), and the Insula. Alternative 3 involves the stimulation (down regulation) of these four targets, and because the inclusion of the tract between the Insula and Orbito-Frontal Cortex, the combination of these three may act as a large “antenna.” The anterior V Coil hits both the OFC and the tract between the OFC and the Insula. The DACG is stimulated by the top Swept-Wing Coil and the left V Coil. The Insula is stimulated by a Swept-Wing Coil on the right. This coil will also function as part an effective three-coil array by contributing to the down-regulation stimulation of the DACG. A sub-alternative is the use of two Swept-Wing Coils on the right to allow for increased stimulation of the Insula. The level of stimulation can be adjusted by changing the power applied from the stimulators to any of the coils.

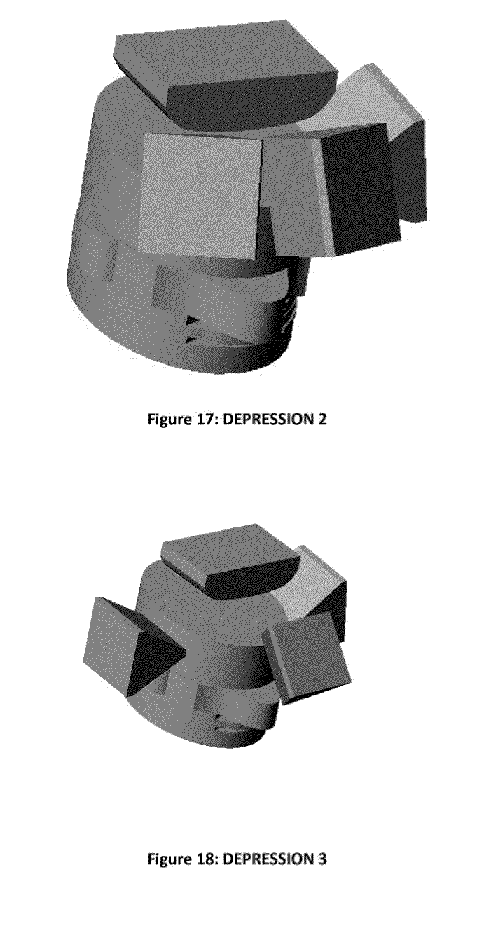

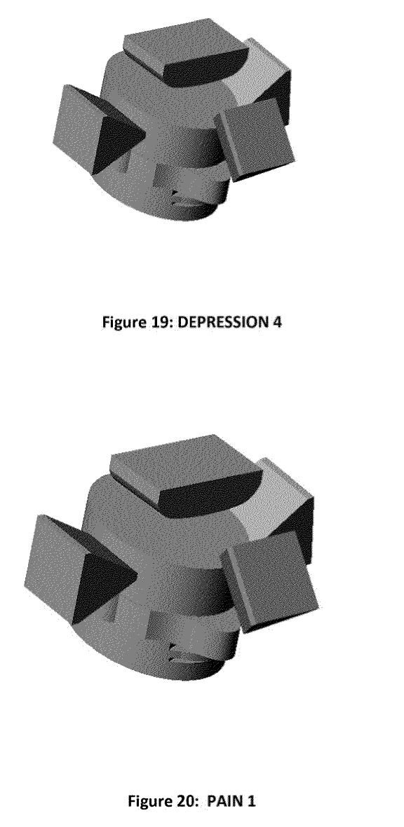

Four alternative coil configurations for depression are included in the table that appears in FIG. 11. As, noted, drawings of these alternatives appear in FIGS. 16, 17, 18, and 20. Alternative 1 (FIG. 16) for depression has magnet configurations with five coils and thus will require five stimulators. In this alternative, the anterior V Coil is used to down-regulate the Right Dorso-Lateral Pre-Frontal Cortex (RDLPFC) or up-regulate Left Dorso-Lateral Pre-Frontal Cortex (LDLPFC). The V-Coil that is positioned on the anterior right side (where x is on the left in this case) is used to stimulate the Orbito-Frontal Cortex and the Subgenual Cingulate. Coils three, four, and five up-regulate the Dorsal Anterior Cingulate Gyrus. This alternative requires five stimulators if one coil per stimulator is used. In alternative 2 (FIG. 17), V Coils one and two are configured as in alternative 1, but only two coils, three and four, are used to up-regulate the DACG. This permits only four stimulators to be used. In alternative 3 (FIG. 18), the anterior V Coil (Coil 1) stimulates (down-regulates) the OFC and the Subgenual Cingulate and the three-coil combination (Coils 2 to 4—two V-Coils lateral to a central-top Swept-Wing Coil) up-regulate the Dorsal Anterior Cingulate Gyrus. In alternative 4 (FIG. 19), Coils 2 to 4 are the same as in alternative 3, but the anterior V-Coil is used to stimulate the Right or Left Dorsal-Lateral Pre-Frontal Cortex, involving down-regulating the RDLPFC or up-regulating the LDLPFC. For the therapy of depression there can be other alternatives, such as stimulation of both the RDLPFC (Down) and LDLPFC (Up). The stimulation of the Insula for addition uses the superior anterior region as the target. Because the Insula has smaller fibers (say relative to the Dorsal Anterior Cingulate Gyms), the power to be applied will be greater.

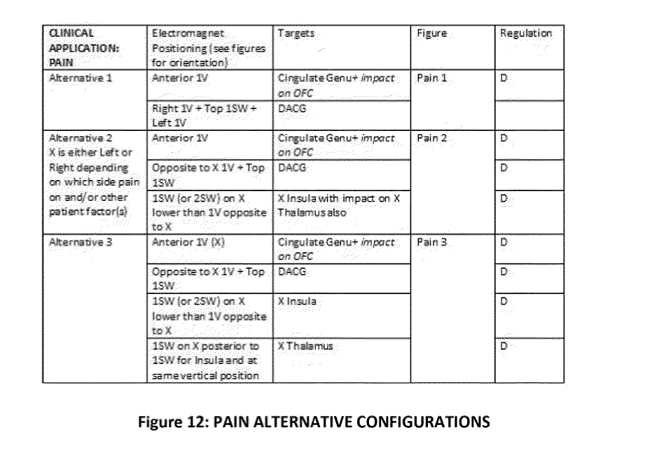

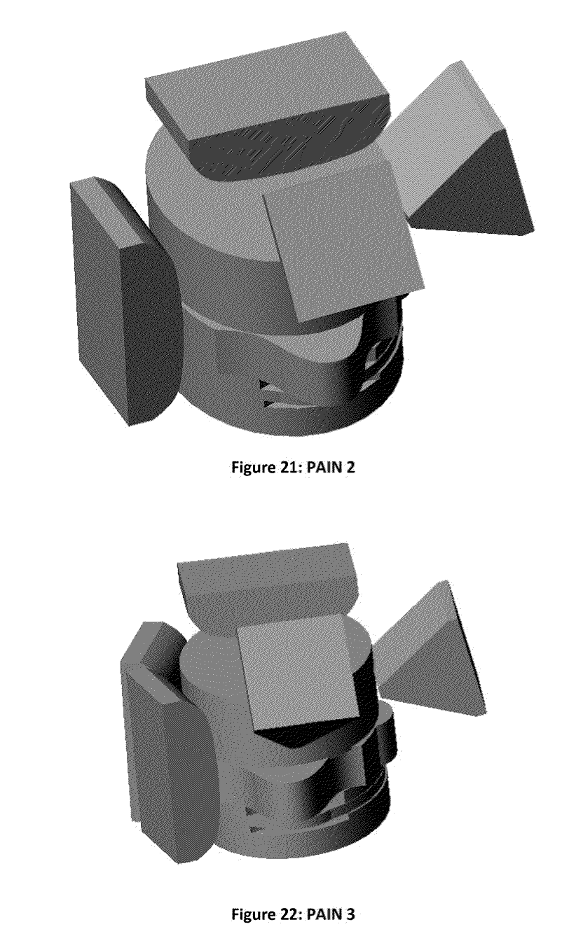

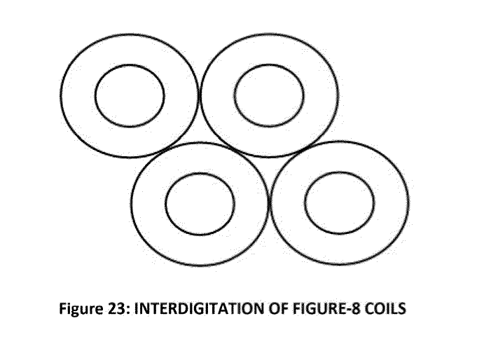

Three alternatives for coil configurations for treatment of pain are included in the table that appears in FIG. 12. As, noted, drawings of these alternatives appear in FIGS. 20, 21, and 22. In alternative 1 (FIG. 20), the anterior V Coil down regulates the Cingulate Genu (and will have some impact on the Orbito-Frontal Cortex as well). The three-coil combination of the top Swept-Wing Coil flanked by lateral V-Coils down regulate the DACG. In alternative 2 (FIG. 21), the DACG is stimulated by the top Swept-Wing Coil and a lateral V-Coil that is placed opposite the side (X) that the Swept-Wing Coil stimulating the Insula is placed. The coil hitting the Insula will have impact on the Thalamus on that side as well. The side X will be patient dependent and may be related to the side of the pain. In alternative 3 (FIG. 22), the same configuration as was used in alternative 2 is employed except that instead of having one Swept-Wing Coil on side X, there are two Swept-Wing Coils, one hitting the Insula and the other the Thalamus. Both coils will impact both of the structures to some extent and both will be impacted more because of the deeper penetration of the two-Swept-Wing Coil configuration.

### General Principles

In applying TMS methods, devices and systems, any of the following general concepts may be applied. For example, in general the TMS systems devices and methods described herein may be configured for aiming and focusing.
V-Coils can be used to shape the field of a Swept-Wing Coil (e.g., using two side V-Coils with current of opposite polarity to a central Swept-Wing Coil will narrow the magnetic field of that central coil)—this may be called a “Triad” configuration
If targets are bilateral, and/or if you need more targets than you can fit magnets in an area, you can choose to stimulate only one of the bilateral targets. This could result in hitting one of the targets A from one side and hitting one of the targets B from other side. In some cases even if the target appears bilaterally in the brain, only one side is application to a given clinical application. For example, only the right Insula is involved in addiction.
In cases where immediate or close-in-time image feedback is available, e.g., PET scan looking at Insula as the target, the results may be used to refine targeting
In cases where immediate or close-in-time physiologic feedback is available, for example, in acute pain, the results can be used to refine targeting

### Array Packing

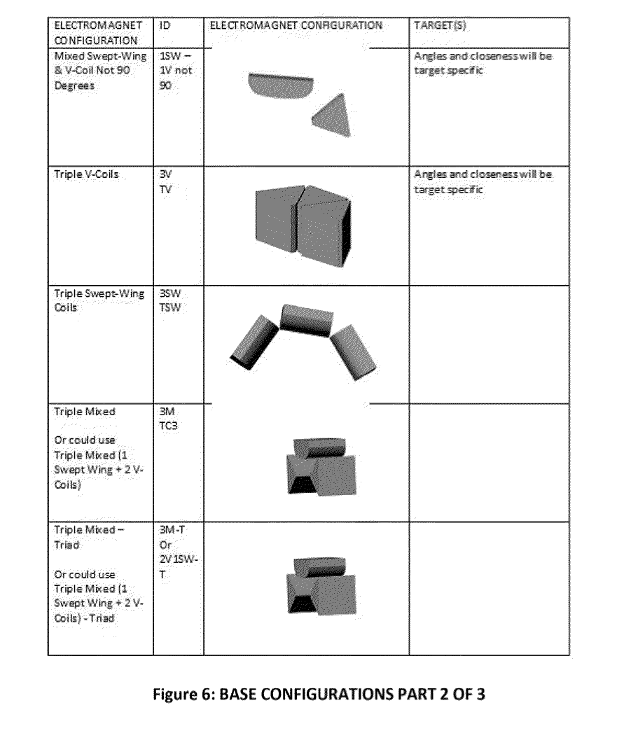

The number of targets that can be regulated simultaneously may depend on the ability to physically place the magnetic coil/magnetic coil arrays
Concentrate on accommodating the needs of the specific target and create target-specific coils where needed
Angles and separations between magnets in array may be target specific
A single coil or array can be configured to hit more than one target depending on how the potential targets are aligned
Incorporate set(s) of coils (up to all the coils for all arrays focusing on various targets) in a single shell to optimize physical coil packing
Physically interdigitate coil sets where appropriate and practical as illustrated by the interdigitation of figure-8 coils shown in FIG. 23.
A coil in one array can provide a function that would normally be provided by a coil in another array (e.g., one of the V-Coils in a Tripled Mixed Configuration (FIG. 6) in an array can be have one of the V-Coils removed and its function at least partially provided by a vertical Swept-Wing Coil in another array as illustrated in the Addiction configuration in FIG. 15.
Can tie stimulation of coil in one array to simultaneous stimulation of a coil in another array using one stimulator instead of requiring two

### Substitutions

Substitution of TMS electromagnet types (e.g., V-Coil versus Swept-Wing Coil) may be driven by physical constraints, and/or power required at the target, and/or wanting to avoid stimulating other structures. Functional effects can be altered by increasing or decreasing power applied given that a V-Coil is more focused but has a weaker magnetic field at a given distance than a Swept-Wing Coil.
   Same or Similar Coil Configurations for More than One Application
The same coils may appear in configurations for two or more clinical applications, but some or all positions may be different or the power applied may be different
More than one application can share the same (set of) configuration(s) aimed at the same targets but have different up/down regulations
A common array configuration may neuro-regulate a common target or set of targets and thus simultaneously treat more than one clinical application. For example, treatment for both addiction and depression can be accomplished by simultaneously down-regulating the DACG and Oribito-Frontal Cortex. In some cases (e.g., chronically depressed addicts), treating two conditions will be beneficial to the patient
Even if a common array configuration is treating more than one clinical application, treatment for one or both can be facilitated by adding an additional target that is not common to both applications
Not all the potential targets for a given condition need to be regulated to treat that condition, but adding simultaneous targets, if practical, can improve the results

### Stimulation

Interleave stimulation of various coil arrays as appropriate to application
Alternative stimulation strategies may be applied to reach the same functional end, for example, depression of neural structures can be accomplished by 1 Hz or lower-frequency stimulation or by theta-burst-pattern stimulation

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of treating a disorder by non-invasive neural stimulation, the method comprising modulating the activity of the majority of a target deep-brain region, wherein the target brain region is selected from the group consisting of: neocortex, medial PFC, LDLPFC, RDLPFC, dorsomedial PFC, ventral PFC, VMPFC, orbitofrontal cortex (OFC), tracts between OFC and insula, cingulate genu, DACG, Pre-Gen. anterior cingulate, subgenual cingulate, posterior cingulate, striatum-DACG connections, tracts between pre-gen, anterior cingulate and insula, insula, amygdala, anterior limb of internal capsule, ventral internal capsule, target, nucleus accumbens, tract between nucleus acumbens and ventral teg., hippocampus, temporal lobes, septum, caudate (nucleus), globus pallidus, anterior nucleus of the thalamus, lateral thalamus, centromedian thalamus, thalamic subregions, subthalamic nucleus, lateral hypothalamic area nuclei, ventromedial nuclei of hypothal., cerebellum, brainstem, and pons.

2. The method of claim 1, wherein the step of modulating the activity comprises up-regulating the activity of the target region.

3. The method of claim 1, wherein the step of modulating the activity comprises down-regulating the activity of the target region.

4. The method of claim 1, wherein the step of modulating the activity comprises non-invasively stimulating the target region to modulate the activity using an array of TMS electromagnets.

5. The method of claim 1, wherein the step of modulating the activity of the target region does not substantially modulate the activity of non-target brain regions.

6. The method of claim 1, wherein the step of modulating the activity comprises applying energy to activate an array of TMS electromagnets.

7. The method of claim 1, further comprising positioning an array of TMS electromagnets to modulate each of the target brain regions.

8. The method of claim 1, wherein the disorder is selected from the group consisting of: addiction, Alzheimer's disease, anogasmia, attention deficit disorder, autism, cerebral palsy, depression, bipolar, depression, unipolar, epilepsy, generalized anxiety disorder, head trauma (acute), hedonism, obesity, OCD, acute pain, chronic pain, Parkinson's disease, persistent vegetative state, phobia, PTSD, social anxiety disorder, rehab/regenesis for post-stroke, post-head trauma, hemorrhagic stroke, ischemic stroke, and Tourette's syndrome.

9. The method of claim 1, further comprising modulating the activity of the majority of a second target brain region.

10. The method of claim 9, wherein the second target brain region is modulated simultaneously with the modulation of the first target brain region.

11. The method of claim 1, wherein the step of modulating the activity is performed by moving at least one TMS coil around a subject's head to stimulate the target brain region.

12. A method of treating a disorder by non-invasive neural stimulation, the method comprising simultaneously modulating the activity of two or more target brain regions to up-regulate or down-regulate activity in the target brain region, wherein the target brain regions are selected from the group consisting of: NeoCortex, Medial PFC, LDLPFC, RDLPFC, Dorsomedial PFC, Ventral PFC, VMPFC, Orbitofrontal Cortex (OFC), Tracts between OFC and Insula, Cingulate Genu, DACG, Pre-Gen. Anterior Cingulate, Subgenual Cingulate, Posterior Cingulate, Striatum-DACG Connections, Tracts between Pre-Gen. Anterior Cingulate and Insula, Insula, Amygdala, Anterior Limb of Internal Capsule, Ventral Internal Capsule, Target, Nucleus Accumbens, Tract between Nucleus Acumbens and Ventral Teg., Hippocampus, Temporal Lobes, Septum, Caudate (Nucleus), Globus Pallidus, Anterior Nucleus of the Thalamus, Lateral Thalamus, Centromedian Thalamus, Thalamic Subregions, Subthalamic Nucleus, Lateral Hypothalamic Area Nuclei, Ventromedial Nuclei of Hypothal., Cerebellum, Brainstem, and Pons.

13. The method of claim 12, wherein the step of simultaneously modulating activity comprises modulating the activity in the majority of the target brain regions.

14. The method of claim 12, wherein the step of simultaneously modulating activity comprises non-invasively stimulating.

15. The method of claim 12, wherein the step of simultaneously modulating activity comprises stimulating by deep-brain TMS.

16. The method of claim 12, wherein the step of simultaneously modulating activity comprises up-regulating activity in one target brain region.

17. The method of claim 12, wherein the step of simultaneously modulating activity comprises down-regulating activity in one target brain region.

18. The method of claim 12, wherein the step of simultaneously modulating activity comprises up-regulating activity in one target brain region while down-regulating activity in another brain region.

19. The method of claim 12, wherein the step of simultaneously modulating activity does not substantially modulate the activity of non-target brain regions.

20. The method of claim 12, wherein the step of simultaneously modulating the activity comprises applying energy to activate one or more arrays of TMS electromagnets.

21. The method of claim 12, further comprising positioning one or more arrays of TMS electromagnets to modulate each of the target brain regions.

22. The method of claim 12, wherein the disorder is selected from the group consisting of: addiction, Alzheimer's disease, anogasmia, attention deficit disorder, autism, cerebral palsy, depression, bipolar, depression, unipolar, epilepsy, generalized anxiety disorder, head trauma (acute), hedonism, obesity, OCD, acute pain, chronic pain, Parkinson's disease, persistent vegetative state, phobia, PTSD, social anxiety disorder, rehab/regenesis for post-stroke, post-head trauma, hemorrhagic stroke, ischemic stroke, and Tourette's syndrome.

23. A method of treating hedonic disorders by non-invasive neural stimulation, the method comprising applying energy to modulate activity of the Orbitofrontal Cortex (OFC).

24. The method of claim 23, wherein the hedonic disorder is selected from the group consisting of: addiction, sexual disorders, and eating disorders.

25. The method of claim 23 wherein the step of applying energy to modulate the activity of the OFC comprises stimulating the OFC to suppress activity.

26. The method of claim 23, wherein the step of applying energy to modulate the activity of the OFC comprises simultaneously stimulating the majority of the OFC.

27. The method of claim 23, wherein the step of applying energy to modulate the activity of the OFC comprises stimulating the OFC without substantially modulating neural activity in other brain regions including those adjacent to the OFC.

28. A method of treating obesity by non-invasive neural stimulation, the method comprising applying energy to modulate the activity of the Orbitofrontal Cortex (OFC).

29. The method of claim 28, wherein the step of applying energy to modulate comprises non-invasively stimulating the OFC with at least one TMS electromagnet.

30. The method of claim 28, wherein the step of applying energy to modulate comprises simultaneously stimulating the majority of the OFC.

31. The method of claim 28, wherein the step of applying energy to modulate comprises stimulating the OFC with an array of TMS electromagnets.

32. The method of claim 28, further comprising arranging an array of TMS electromagnets to target the OFC.

33. The method of claim 28, further comprising emitting energy from an array of TMS electromagnets to focus the emitted energy on the OFC.

34. The method of claim 28, wherein the step of applying energy to modulate comprises inhibiting or suppressing activity of the OFC.

35. A method of treating addiction by non-invasive neural stimulation, the method comprising simultaneously applying energy to modulate the activity of two or more target brain regions selected from the group consisting of: the insula, the Orbitofrontal Cortex (OFC), the tracts between the OFC and the Insula, and the DACG.

36. A method of treating depression by non-invasive neural stimulation, the method comprising simultaneously applying energy to modulate the activity of two or more target brain regions selected from the group consisting of: the LDLPFC, the RDLPFC, the DACG, and the Orbitofrontal Cortex (OFC).

37. A method of treating pain by non-invasive neural stimulation, the method comprising simultaneously applying energy to modulate the activity of two or more target brain regions selected from the group consisting of: the Cingulate Gyms, the DACG, the Insula and the Lateral Thalamus.

38. The method of any of claim 35, wherein the step of simultaneously applying energy to modulate the activity comprises non-invasively stimulating using at least one TMS electromagnet.

39. The method of any of claim 36, wherein the step of simultaneously applying energy to modulate the activity comprises non-invasively stimulating using at least one TMS electromagnet.

40. The method of any of claim 37, wherein the step of simultaneously applying energy to modulate the activity comprises non-invasively stimulating using at least one TMS electromagnet.

41. The method of any of claim 35, wherein the step of simultaneously applying energy to modulate the activity comprises simultaneously stimulating the majority of the target brain regions.

42. The method of any of claim 36, wherein the step of simultaneously applying energy to modulate the activity comprises simultaneously stimulating the majority of the target brain regions.

43. The method of any of claim 37, wherein the step of simultaneously applying energy to modulate the activity comprises simultaneously stimulating the majority of the target brain regions.

44. The method of any of claim 35, wherein the step of simultaneously applying energy to modulate activity comprises stimulating by TMS using a plurality of TMS electromagnets.

45. The method of any of claim 36, wherein the step of simultaneously applying energy to modulate activity comprises stimulating by TMS using a plurality of TMS electromagnets.

46. The method of any of claim 37, wherein the step of simultaneously applying energy to modulate activity comprises stimulating by TMS using a plurality of TMS electromagnets.

47. The method of any of claim 35, wherein the step of simultaneously applying energy to modulate activity comprises up-regulating activity in one target brain region.

48. The method of any of claim 36, wherein the step of simultaneously applying energy to modulate activity comprises up-regulating activity in one target brain region.

49. The method of any of claim 37, wherein the step of simultaneously applying energy to modulate activity comprises up-regulating activity in one target brain region.

50. The method of any of claim 35, wherein the step of simultaneously applying energy to modulate activity comprises down-regulating activity in one target brain region.

51. The method of any of claim 36, wherein the step of simultaneously applying energy to modulate activity comprises down-regulating activity in one target brain region.

52. The method of any of claim 37, wherein the step of simultaneously applying energy to modulate activity comprises down-regulating activity in one target brain region.

53. The method of any of claim 35, wherein the step of simultaneously applying energy to modulate activity comprises up-regulating activity in one target brain region while down-regulating activity in another brain region.

54. The method of any of claim 36, wherein the step of simultaneously applying energy to modulate activity comprises up-regulating activity in one target brain region while down-regulating activity in another brain region.

55. The method of any of claim 37, wherein the step of simultaneously applying energy to modulate activity comprises up-regulating activity in one target brain region while down-regulating activity in another brain region.

56. The method of any of claim 35, wherein the step of simultaneously applying energy to modulate activity does not substantially modulate the activity of non-target brain regions.

57. The method of any of claim 36, wherein the step of simultaneously applying energy to modulate activity does not substantially modulate the activity of non-target brain regions.

58. The method of any of claim 37, wherein the step of simultaneously applying energy to modulate activity does not substantially modulate the activity of non-target brain regions.

59. The method of any of claim 35, further comprising positioning one or more arrays of TMS electromagnets to modulate each of the target brain regions.

60. The method of any of claim 36, further comprising positioning one or more arrays of TMS electromagnets to modulate each of the target brain regions.

61. The method of any of claim 37, further comprising positioning one or more arrays of TMS electromagnets to modulate each of the target brain regions.

62. A method of treating a disorder by targeted deep-brain Transcranial Magnet Stimulation of a neuronal circuit associated with the disorder, the method comprising:
identifying a plurality of target brain regions from a neuronal circuit associated with the neuronal disorder;
aiming a plurality of TMS electromagnets at each of the target brain regions,
wherein at least one of the target brain regions comprises a deep brain target; and
applying power to the TMS electromagnets to modulate the activity of the target brain regions.

63. The method of claim 62, wherein the disorders treated are selected from the group consisting of: addiction, Alzheimer's disease, anogasmia, Attention Deficit Disorder, autism, cerebral palsy, bipolar depression, unipolar depression, epilepsy, generalized anxiety disorder, head trauma (acute), hedonism, obesity, OCD, acute pain, chronic pain, Parkinson's disease, persistent vegetative state, phobia, PTSD, social anxiety disorder, post-stroke and post-heat trauma rehabilitation/regenesis, Hemorrhagic stroke, ischemic stroke, and Tourette's syndrome.

64. The method of claim 62, wherein the step of aiming comprises simultaneously aiming the TMS electromagnets at each of the target brain regions.

65. The method of claim 62, wherein the step of aiming comprises determining positions and orientation for each of the plurality of TMS electromagnets based on the target and one or more of an atlas, brain imaging, or external landmarks on head.

66. A method of treating addiction by targeted deep-brain Transcranial Magnet Stimulation, the method comprising:
targeting one or more TMS electromagnets at each of two or more of the targets selected from the list comprising: the Orbitofrontal Cortex (OFC), the tracts between the OFC and the Insula, the DACG, the Medial PFC, the Striatum-DACG connections, the anterior limb of the internal capsule, and the Insula; and
applying power to the one or more TMS electromagnets to modulate the activity of the targeted brain regions to treat addiction.

67. A method of treating obesity by targeted deep-brain Transcranial Magnet Stimulation, the method comprising:
targeting one or more TMS electromagnets at each of two or more of the targets selected from the list comprising: the Orbitofrontal Cortex (OFC), the Insula, the lateral hypothalamic area nuclei, and the ventromedial nuclei of the hypothalamus; and
applying power to the one or more TMS electromagnets to modulate the activity of the targeted brain regions to treat obesity.

68. A method of treating pain by targeted deep-brain Transcranial Magnet Stimulation, the method comprising:
targeting one or more TMS electromagnets at each of two or more of the targets selected from the list comprising: the cingulate gyrus, the DACG, the Insula and the lateral thalamus; and
applying power to the one or more TMS electromagnets to modulate the activity of the targeted brain regions to treat pain.

69. A method of treating depression by targeted deep-brain Transcranial Magnet Stimulation, the method comprising:
targeting one or more TMS electromagnets at each of two or more of the targets selected from the list comprising: the LDLPFC, the RDLPFC, the Orbitofrontal Cortex (OFC), and the subgenual cingulate; and
applying power to the one or more TMS electromagnets to modulate the activity of the targeted brain regions to treat depression.
